(19) United States
(12) Patent Application Publication
(10) Pub. No.: US 2020/0140483 A1
(43) Pub. Date: May 7, 2020

(54) USE OF Y PEPTIDE IN PREPARATION OF DRUG OR HEALTHCARE PRODUCT FOR LOWERING BLOOD PRESSURE

(71) Applicant: WITHYOU BIOTECHNOLOGY CO., LTD., GUANGZHOU, GUANGDONG (CN)

(72) Inventors: Wuguang PAN, GUANGZHOU, GUANGDONG (CN); WEI ZHU, GUANGZHOU, GUANGDONG (CN)

(21) Appl. No.: 16/628,917

(22) PCT Filed: Feb. 11, 2018
(86) PCT No.: PCT/CN2018/076277

§ 371 Date: Jan. 6, 2020

(30) Foreign Application Priority Data
Jul. 7, 2017 (CN) 201710550021.4

(51) Int. Cl. C07K 5/06

(52) U.S. Cl. CPC C07K 5/06; A61K 38/00

(57) ABSTRACT

Disclosed is use of a YY-dipeptide in the manufacture a medicament or a healthcare product for lowering blood pressure. In the present disclosure, ACE inhibitory activities of dipeptides are virtually screened by means of a molecular docking method and a self-developed software, based on the determined crystal structure of ACE enzyme. The dipeptides obtained by the virtual screening were experimentally verified for the ACE inhibitory activity thereof. As a result, the YY-dipeptide is found to have relatively good ACE inhibitory activity. A peptide or protein composed of multiple tyrosine residues can be hydrolyzed into an active YY-peptide in vivo, and produce the same or a similar effect.

### CROSS-REFERENCE TO RELATED APPLICATIONS

This application is based upon and claims priority to Chinese Patent Application No. 201710550021.4, filed on Jul. 7, 2017, the entire contents of which are incorporated herein by reference.

### TECHNICAL FIELD

The present disclosure relates to new use of a peptide, and particularly to use of Y-peptide in the preparation a medicament or a healthcare product for lowering blood pressure.

### BACKGROUND

Angiotensin converting enzyme (ACE) is a zinc metalloproteinase, a carboxyl-dipeptidase, and is one of the important proteases involved in the renin-angiotensin system. The ACE plays an important role in the regulation of human blood pressure. It converts angiotensin I to angiotensin II by removing His-Leu at the terminal of angiotensin I. Angiotensin II allows contraction of arterial smooth muscle, resulting in rapidly increased blood pressure. An effective approach of lowering blood pressure is inhibiting the activity of ACE. Current pharmaceuticals for treatment of hypertension are mostly synthetic chemicals, which have certain adverse effects, such as cough, taste disorders, rashes and the like. Thus, ACE-inhibitory peptides, which are prepared from food-borne proteins as raw material, represent an important direction for the development of antihypertensive pharmaceuticals due to high level of safety, low level of toxic- or side-effects, and other advantages thereof.

Short peptides are easy to prepare, and substantially have no side effects on human bodies. Researches have showed that short peptides with specific structures, for example, dipeptides, tripeptides and tetrapeptides, have certain inhibitory effects on the activity of ACE, and thus can be quite promising ACE enzyme inhibitors.

It has reported a model of structure-activity relationship for angiotensin converting enzyme dipeptide inhibitors was established from the primary structures of peptide chains, by taking a molecular electro-negativity edge vector (MEEV) as a parameter, and taking 36 angiotensin converting enzyme dipeptide inhibitors as samples. With the model analysis, a rule of “distance of two, five and seven chemical bonds” for dipeptide bonds inhibiting enzyme activity was obtained. The rules were: (1) the carboxyl group of the peptide bond formed a two-ligand with a Zinc atom, which is stabilized by the hydrogen bond formed between the Nitrogen atom and the carboxyl oxygen of the peptide bond; (2) a five-bond structure unit was formed between the carboxylate radical, which was lined with Arg (Arginine) with positive charged salt bond in the ACE enzyme, and the amino group of the second amino acid, to play a key role in antihypertensive effect; and (3) the amino group of the peptide bond in the dipeptide inhibitor containing an aromatic amino acid showed a trans configuration with the hydroxyl terminal of the benzene ring, with seven bonds therebetween.

Further, it has reported a multiple linear regression (MLR) model of structure and activity was established by characterizing the sequences of dipeptide, tripeptide and tetrapeptide competitively inhibiting Angiotensin Converting Enzyme (ACE), respectively, by using an amino acid structure describer SVHEHS. The correlation coefficient, cross validation correlation coefficient, root mean square error and external validation correlation coefficient are respectively 0.851, 0.781, 0.327, and 0.792 for the ACE inhibitory dipeptide model; respectively 0.805, 0.717, 0.339, and 0.817 for the tripeptide model; and respectively 0.792, 0.553, 0.393, and 0.630 for the tetrapeptide model.

Further, it has reported a model for structure-activity relationship of dipeptide inhibitors of angiotensin converting enzyme was established by taking molecular electro-negativity edge vector (MEEV) as a parameter, and taking 36 angiotensin converting enzyme dipeptide inhibitors as samples. The model analysis showed that hydrophobic amino acids, for example, aromatic amino acids and branched-chain amino acids at C-terminal, were key factors affecting inhibitory activity for ACE.

Further, it has reported that the hydrophobicity (X15), electrical property (X17), and stereoscopic feature (X24) of C-terminal amino acid, as well as stereoscopic feature (X12) of N-terminal amino acid of the dipeptide were highly correlated to the activity of the peptide.

Further, it has demonstrated a dipeptide model with R²=0.851, RMSE=0.327, Q^{2LOO}=0.781, Q^{2ext}=0.792, and the hydrophobicity and charge property of the C-terminal amino acid and the stereoscopic property of the N-terminal amino acid had a relatively strong influence on the activity of the ACE inhibitory dipeptide. Particularly, the strong hydrophobicity and weak charge property of the C-terminal amino acid had a positive effect on the activity of an ACE inhibitory dipeptide; and the hydrophobicity, electrical property, and stereoscopic property of C-terminal amino acid, and the stereoscopic property of N-terminal amino acid were highly correlated with the activity of the peptide.

In prior art, short peptides inhibiting ACE activity were researched from multiple perspectives in an attempt to determine the relationship between the structure of a short peptide and ACE inhibitory activity. However, the existing research results have limitations in that the accuracy of the predicted results is low, and a dipeptide with high ACE inhibitory activity has not been found.

Therefore, it is of great practical significance to develop a dipeptide with high inhibitory activity.

### SUMMARY

One object of the present disclosure is to provide use of a Y-peptide in preparing a medicament or a healthcare product for lowering blood pressure.

The technical solutions adopted in the present disclosure are as follows.

According to the present disclosure, ACE inhibitory activities of 400 dipeptides were virtually screened by means of a molecular docking method and a self-developed software, based on the determined crystal structure of ACE enzyme. The dipeptides obtained by the virtual screening were experimentally verified for the ACE inhibitory activity thereof. As a result, a YY-peptide was found to have a calculated score of 117.903, and have excellent ACE inhibitory activity and can significantly reduce blood pressure. A peptide or protein composed of multiple tyrosine (Tyr/Y) residues can be hydrolyzed into an active YY-peptide in vivo, and produce the same or similar effect. The YY-peptide, as well as the peptide or protein composed of multiple tyrosine (Tyr/Y) residues, are expected to be developed into a medicament or a healthcare product for lowering blood pressure. In addition, the YY-peptide can be used for preparing an ACE inhibitor, especially an ACE inhibitor for using in experiments.

Use of a Y-peptide in the manufacture of a medicament or a healthcare product for lowering blood pressure is provided, in which a general formula of the Y-peptide is Yn, wherein n may be an integer no less than 2, and Y may be of L-type or D-type.

Use of a Y-peptide in the manufacture of an inhibitor of ACE enzyme activity is provided, in which a general formula of the Y-peptide is Yn, wherein n may be an integer no less than 2, and Y may be of L-type or D-type.

Specially, n may be an integer ranging from 2 to 100 for synthesis economy and other concerns.

At least one amino acid in the Y-peptide can optionally modified with a group for improving in vivo stability of the Y-peptide.

The Y-peptide may also include a simple derivative thereof which is pharmaceutically or bromatologically acceptable. The simple derivative may include, but not limited to a medicinal salt and ester of the dipeptide. The medicinal salt may include, but not limited to potassium, calcium, sodium, zinc, iron and ferrous salts, as well as tartrate and succinate. The medicinal ester may include, but not limited to C2-C10 medicinal esters.

The Y-peptide of the present disclosure, especially the YY peptide, can significantly inhibit ACE activity and has better effect in lowering blood pressure. At the same time, the YY peptide can regulate metabolism and thus have double effect in lowering blood pressure. Thus, the Y-peptide of the present disclosure is especially suitable for development into a healthcare product for lowering blood pressure.

### DETAILED DESCRIPTION

According to the present disclosure, ACE inhibitory activities of 400 dipeptides were virtually screened by means of a molecular docking method and a self-developed software, based on the determined crystal structure of ACE enzyme. The dipeptides obtained by the virtual screening were experimentally verified for the ACE inhibitory activity thereof. As a result, a YY-peptide was found to have excellent ACE inhibitory activity and can significantly reduce blood pressure. A peptide or protein composed of multiple tyrosine (Tyr/Y) residues can be hydrolyzed into an active YY-peptide in vivo, and produce the same or similar effect. The YY-peptide, as well as the peptide or protein composed of multiple tyrosine residues, are expected to be developed into a medicament or a healthcare product for lowering blood pressure. In addition, the YY-peptide can be used for preparing an ACE inhibitor, especially an ACE inhibitor for using in experiments.

ACE Enzyme Activity Experiments

Experimental results showed that the half maximal inhibitory concentration of an YY-peptide for ACE enzyme activity was 29.7 μM.

The experimental results proved that the YY-peptide could significantly inhibit the ACE enzyme activity, had a good potential for lowering blood pressure. Thus, the YY-peptide was expected to be developed into a medicament or a healthcare product for lowering blood pressure.

Animal Experiments

Method: SHR rats were randomly divided into a model control group, a positive control group and an YY-peptide group; and SD rats were randomly divided into a normal control group 1 and a normal control group 2, with 10 rats in each group, and half male and half female. Each group was intragastrically administrated a solution of a corresponding tested substance or ultrapure water, in a volume of 10 mL/kg body weight, once per day for 30 consecutive days. The blood pressure and heart rates of the animals were measured during the administration of the tested substance.

The blood pressure was measured with an LE5002 noninvasive blood pressure meter.

Experimental results: The data obtained on the 7^{th} day: the blood pressure of the DD water group (model control group) was 161±6, the blood pressure of the captopril group (10 mg/kg) was 156±6, and the blood pressure of the YY-peptide group (10 mg/kg) was 157±7. The data showed that the YY-peptide had significant effect in lowering blood pressure, which was basically equivalent to that of captopril. While, the YY-peptide had no obvious effect on body weight and heart rates of the rats.

Both the ACE enzyme activity and the rat animal experiments show that the YY-peptide had in vivo significant effect in lowering blood pressure. The YY-peptide, as well as the peptide or protein composed of multiple tyrosine residues, are expected to be developed into a medicament or a healthcare product for lowering a blood pressure. In addition, the YY-peptide can be used for preparing an ACE enzyme inhibitor, especially an ACE enzyme inhibitor for experiments.

1-10. (canceled)

11. A method for treatment or improvement of hypertension, or for inhibition of Angiotensin Converting Enzyme (ACE) activity, comprising administering a therapeutically effective amount of a Y-peptide to a subject, wherein the Y-peptide has a general formula of Yn, in which n is an integer no less than 2, preferably n is an integer ranging from 2 to 100; and Y is of L- or D-type.

12. The method according to claim 11, wherein the Y-peptide is modified, at least one amino acid, with a group for improving in vivo stability of the Y-peptide.

13. The method according to claim 11, wherein the Y-peptide comprises a simple derivative thereof which is pharmaceutically or bromatologically acceptable.

14. The method according to claim 13, wherein the simple derivative is a medicinal salt or ester of the Y-peptide.

15. The method according to claim 14, wherein the medicinal salt is one of potassium, calcium, sodium, zinc, iron, ferrous, tartrate and succinate salts of the Y-peptide;
wherein the medicinal ester is a C2-C10 medicinal ester of the Y-peptide.

16. A composition for treatment or improvement of hypertension, or for inhibition of Angiotensin Converting Enzyme (ACE) activity, comprising a Y-peptide as an active ingredient, wherein the Y-peptide has a general formula of Yn, in which n is an integer no less than 2, preferably n is an integer ranging from 2 to 100; and Y is of L- or D-type.

17. The composition according to claim 16, wherein the Y-peptide is modified, at least one amino acid, with a group for improving in vivo stability of the Y-peptide.

18. The composition according to claim 16, wherein the Y-peptide comprise a simple derivative thereof which is pharmaceutically or bromatologically acceptable.

19. The composition according to claim 18, wherein the simple derivative is a medicinal salt or ester of the Y-peptide.

20. The composition according to claim 19, wherein the medicinal salt is one of potassium, calcium, sodium, zinc, iron, ferrous, tartrate and succinate salts of the Y-peptide;
wherein the medicinal ester is a C2-C10 medicinal ester of the Y-peptide.
